(19) United States
(12) Patent Application Publication
(10) Pub. No.: US 2026/0041576 A1
(43) Pub. Date: Feb. 12, 2026

(54) HALLUX CORRECTOR

(71) Applicant: Guangzhou New Design Biotechnology Co., Ltd, Guangzhou (CN)

(72) Inventors: Shaowei ZHANG, Guangzhou (CN); Sile ZOU, Guangzhou (CN); Jiehong LIN, Guangzhou (CN); Yuhui OU, Guangzhou (CN)

(73) Assignee: Guangzhou New Design Biotechnology Co., Ltd, Guangzhou (CN)

(21) Appl. No.: 18/799,509

(22) Filed: Aug. 9, 2024

(51) Int. Cl. A61F 5/01

(52) U.S. Cl. CPC A61F 5/019; A61F 2005/0146; A61F 2005/0165

(57) ABSTRACT

This disclosure provides a hallux corrector, including: a foot side support member configured to be worn on a sole; a hallux support member configured to be worn on a hallux, where the hallux support member is rotatably connected to one end of the foot side support member; a push member, including a push plate as well as a stud and a limit guide member that are vertically arranged on the push plate; and a knob, where the knob includes a threaded sleeve, and the threaded sleeve is screwed to the stud. The limit guide member slidably penetrates into the foot side support member. The threaded sleeve is defined at one side of the foot side support member. When the threaded sleeve is rotated, the stud moves axially relative to the threaded sleeve, the foot side support member and the hallux support member as a whole to the push plate changes.

### TECHNICAL FIELD

This disclosure relates to the technical field of medical auxiliary instruments, in particular to a hallux corrector.

### BACKGROUND

In the field of medical auxiliary instruments, the correction and treatment of foot deformity, especially toe deformity, has always been the focus of research. At present, the most common toe deformity is hallux deviation, where a hallux is bent towards one side of an adjacent toe, causing squeezing, which is likely to cause pain during walking, cause dermatophytosis due to bacteria breeding, or the like. The hallux deformity needs to be corrected by a hallux corrector, so that the hallux returns to a correct straightening direction. A common hallux corrector in the industry includes a foot side support plate, a hallux support plate rotatably connected to the foot side support plate, and a deviation correction adjusting structure arranged at a joint between the foot side support plate and the hallux support plate. The deviation correction adjusting structure is configured to drive the hallux support plate to move laterally relative to a foot body, that is, to drive the hallux to leave from an adjacent toe by a certain distance, so as to realize correction adjustment. In an existing hallux corrector, a deviation correction adjusting structure has a push rod structure, that is, a push rod is arranged to move relative to a foot side support plate and a hallux support plate. The push rod abuts against one side of a foot body, and then the push rod is driven to act, so that the foot side support plate and the hallux support plate connected to the push rod move along the push rod, and this can be regarded as adjustment of a tension degree of the foot side support plate and the hallux support plate relative to the foot body. Since the foot side support plate is fixedly bundled on the foot body, a degree of freedom of movement of the foot side support plate depends on the tightness of a bandage, and a degree of freedom of swing of the foot side support plate is small. However, the hallux has a certain degree of freedom of lateral swing, so the hallux and the hallux support plate can integrally move laterally. Therefore, during adjustment, a tension force mainly acts on a hallux end, which drives the hallux support plate to move laterally together with the hallux, thereby realizing lateral movement distance adjustment for the hallux.

With the continuous progress of medical technology, people have imposed increasingly high requirements on toe correctors, hoping that the toe correctors can not only provide effective correction effects but also ensure the comfort and use experience of patients. At present, there are various types of toe correctors on the market, but most of them have the problems of fixed structure and poor adaptability. These correctors often cannot be accurately adjusted according to toe deformity degrees and individual differences of the patients, resulting in poor correction effects or discomfort during use. In addition, some correctors have certain adjustment accuracy, but they are stuck and therefore cannot slide smoothly during adjustment. For example, a deviation correction adjusting structure of a hallux corrector in the industry is designed to adopt a threaded knob structure, in which a screw cap is in thread fit with a push rod. By rotating the screw cap, the push rod is forced to move up and down. In this threaded knob structure, a threaded hole is formed in the center of a lifting rod, and a peripheral wall of the lifting rod is provided with a longitudinal guide protrusion or guide groove. The guide protrusion or guide groove matches a corresponding guide groove or guide protrusion on a foot side support plate or a hallux support plate, so that when the screw cap rotates, the push rod does not rotate with the screw cap, and the push rod moves up and down along the guide groove. However, such threaded knob structure requires high processing fit accuracy between the guide protrusion and the guide groove, otherwise it is likely to cause shake in a circumferential direction or unsmooth up-down sliding, or even cause phenomena such as stuck during adjustment, which affects the use experience of the patients.

Therefore, designing a type of hallux corrector which is easy to process and unlikely to get stuck during operation has become an urgent problem to be solved in the technical field of medical auxiliary instruments.

### SUMMARY

In view of the above description, this disclosure provides a hallux corrector which is easy to process, unlikely to get stuck during operation, and also capable of ensuring the stability and comfort during correction, thereby providing personalized correction schemes for patients.

This disclosure adopts the following technical solution.

A hallux corrector includes:
a foot side support member, capable of being worn on a sole and also capable of abutting against one side of the sole;
a hallux support member, capable of being worn on a hallux and also capable of abutting against an outer side surface of the hallux, where the hallux support member is rotatably connected to one end of the foot side support member;
a push member, where the push member includes a push plate, a stud, a limit guide member, and a knob; the stud and the limit guide member are arranged on the push plate; and
the knob is rotatably mounted at a rotary joint between the foot side support member and the hallux support member; the knob includes a threaded sleeve; the threaded sleeve is screwed to the stud; the limit guide member slidably penetrates into the foot side support member; the push plate and the threaded sleeve are respectively located at two sides of the foot side support member; the push plate abuts against a metatarsophalangeal joint of a foot side; a position of the threaded sleeve is defined at one side of the foot side support member through the rotary threaded sleeve; when the stud moves axially relative to the threaded sleeve, a distance from the threaded sleeve, the foot side support member and the hallux support member as a whole to the push plate changes; and the hallux support member, together with the hallux, changes in position relative to the push plate, so as to correct a position of the hallux.

This disclosure has the following beneficial effects.

Compared with the prior art, the hallux corrector of this disclosure adopts the limit guide member that slidably penetrates into the guide hole in the foot side support member, so that it is unnecessary to adopt a guide protrusion and guide groove fitting manner, facilitating processing and preventing from being get stuck during use. In addition, a connecting shaft is provided to connect the threaded sleeve and the stud, the stud slidably sleeves the connecting shaft, and the stud is ensured not to be separated from the threaded sleeve.

### BRIEF DESCRIPTION OF THE DRAWINGS

The accompanying drawings are intended to provide a further understanding of this disclosure and constitute a part of the specification, and are intended to interpret this disclosure together with the following specific embodiments without constituting any limitation on this disclosure. In the accompanying drawings:

FIG. 1 is a three-dimensional assembly diagram of a hallux corrector of this disclosure;

FIG. 2 is an exploded view of the hallux corrector of this disclosure from a first perspective;

FIG. 3 is an exploded view of the hallux corrector of this disclosure from a second perspective;

FIG. 4 is a cross-sectional view of the hallux corrector of this disclosure;

FIG. 5 is a reference diagram of an operating state of the hallux corrector of this disclosure;

FIG. 6 is a schematic diagram of states of the hallux corrector of this disclosure using different hallux support members;

FIG. 7 is a structural diagram of a push member of another embodiment of the hallux corrector of this disclosure; and

FIG. 8 is an assembly diagram of a foot side support member of the hallux corrector of this disclosure when equipped with the push member shown in FIG. 7.

### DETAILED DESCRIPTION OF THE EMBODIMENTS

Specific embodiments of this disclosure are described below in detail with reference to the accompanying drawings. It should be understood that the specific embodiments described herein are merely used to describe and explain this disclosure rather than to limit this disclosure.

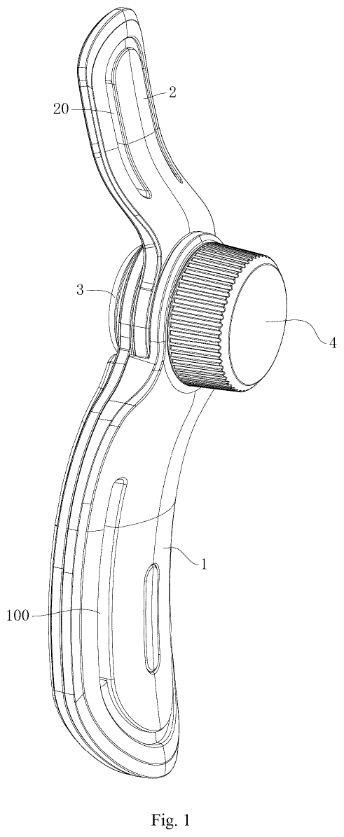

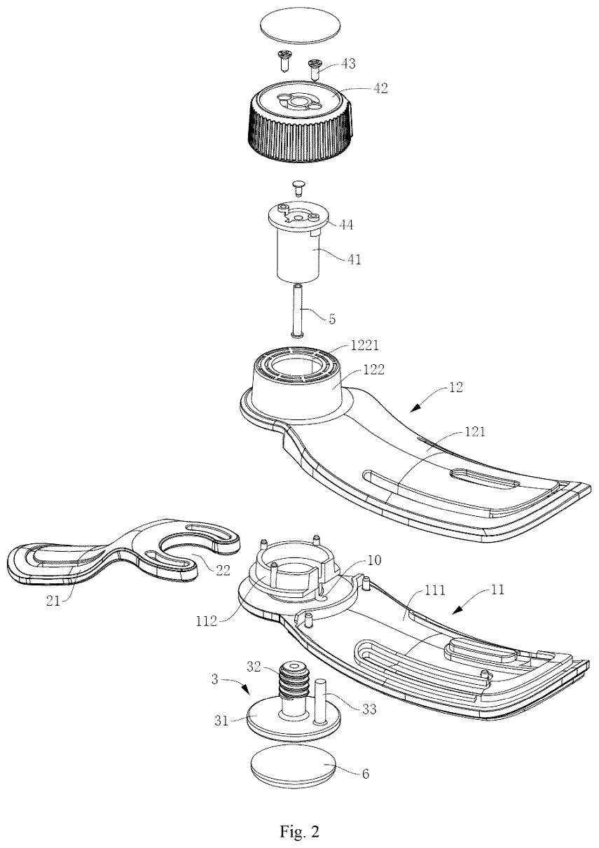

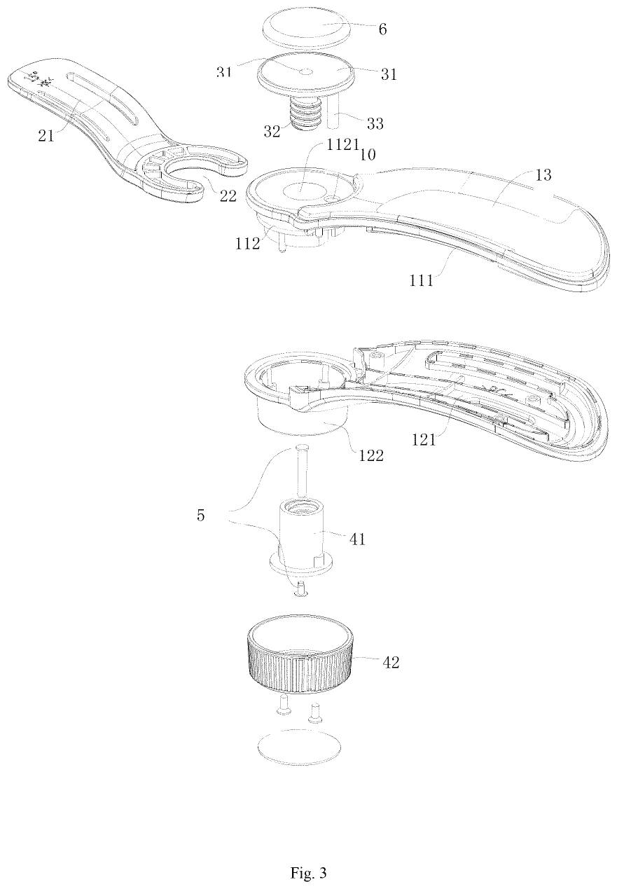

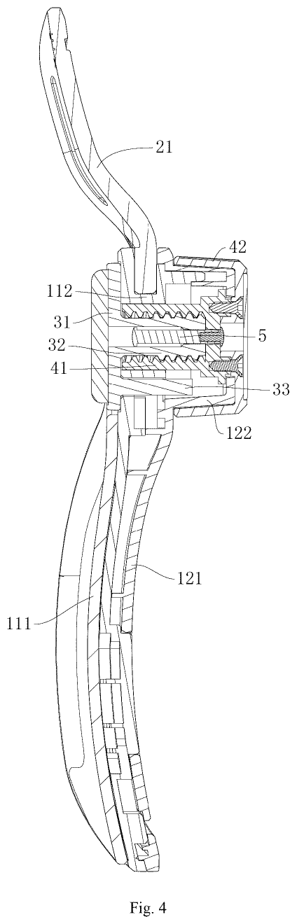

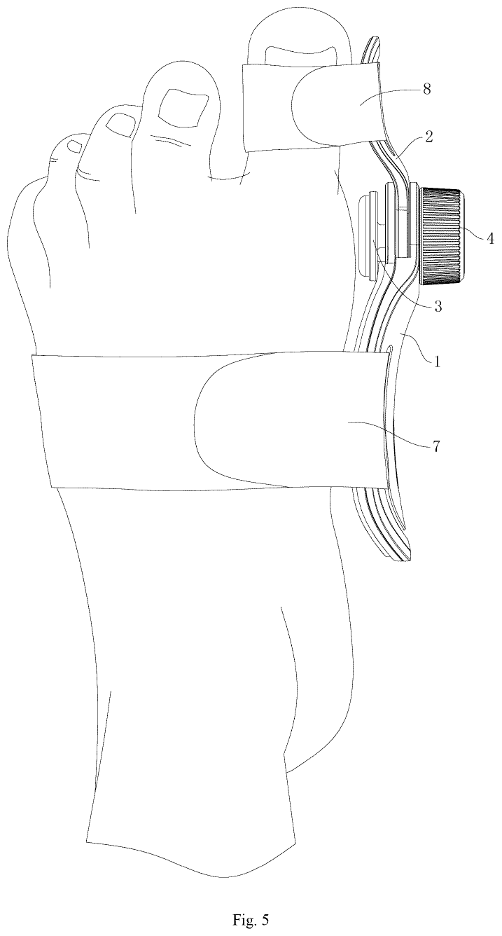

As shown in FIG. 1 to FIG. 5, this disclosure provides a hallux corrector, including a foot side support member 1, a hallux support member 2, a push member 3, and a knob 4. The foot side support member 1 is rotatably connected to the hallux support member 2. The foot side support member 1 is configured to be worn on a sole so as to abut against one side of the sole. The hallux support member 2 is configured to be worn on a hallux and abut against an outer side surface of the hallux. The push member 3 includes a push plate 31 abutting against a metatarsophalangeal joint of a foot side, as well as a stud 32 and a limit guide member 33 that are vertically arranged on the push plate 31. The knob 4 is rotatably mounted at a rotary joint between the foot side support member 1 and the hallux support member 2. The knob 4 includes a threaded sleeve 41. The threaded sleeve 41 has an internal thread and is screwed to the stud 32. The foot side support member 1 is provided with a guide hole 10, and the limit guide member 33 slidably penetrates into the guide hole 10 of the foot side support member 1. The push plate 31 and the knob 4 are respectively located at two sides of the foot side support member 1, so that the knob 4 is rotated to drive the stud 32 and the push plate 31 to slide along a sliding direction of the limit guide member 33, so as to adjust the position, thereby adjusting a force of the push plate 31 abutting against the metatarsophalangeal joint.

The hallux corrector of this disclosure can accurately correct hallux deformity by cooperation of the foot side support member 1 and the hallux support member 2. The foot side support member 1 abuts against the metatarsal bone of the sole to provide a stable support foundation for the corrector, and the hallux support member 2 is close to the outer side surface of the hallux, ensuring that a corrective force can accurately act on key positions. In addition, the design of the push member 3 allows a user to accurately adjust an abutting force of the push plate 31 against the metatarsophalangeal joint by rotating the knob 4, thereby realizing personalized accurate correction and improving the correction effect. No matter users with different foot shapes, users with different deformity degrees, or users of different ages, they can find the most suitable correction state through simple adjustment. This characteristic of strong personalized adaptability greatly improves the use effect and user experience of the corrector.

The hallux corrector of this disclosure is designed with sufficient consideration of the comfort of the user. Both the foot side support member 1 and the hallux support member 2 can adopt an ergonomic design, which can fit the shapes of the sole and the hallux, thereby reducing friction and pressure. In addition, the push member 3 and the knob 4 are easy and convenient to operate, without bringing additional trouble to the user. Such high-comfort design allows the user to wear the corrector for a long time, thereby accelerating the correction process.

The hallux corrector of this disclosure features compact structure and simple operation. Users can wear and adjust the corrector with just a few simple steps without professional assistance. In addition, the corrector is small in size and convenient to carry, so users can wear it at any time in daily life, regardless of time and place restrictions.

In order to prevent the push member 3 from coming out of the threaded sleeve 41 when the knob 4 is rotated, the hallux corrector further includes a connecting shaft 5, the connecting shaft 5 penetrates into the threaded sleeve 41 and the stud 32, the stud 32 slidably sleeves the connecting shaft 5, and two ends of the connecting shaft 5 are each provided with a limiting part 51 to limit the position in an axial direction, so as to prevent the stud 32 and the threaded sleeve 41 from leaving each other.

In a preferred embodiment of this disclosure, the threaded sleeve 41 includes a sleeve portion and an end wall portion formed at one end of the sleeve portion. One end of the connecting shaft 5 extends into the sleeve portion and is fixedly connected to the end wall portion. A sleeve hole 311 is provided in a position of the push plate 31 corresponding to a center axis of the stud 32. The sleeve hole 311 extends close to a tail end of the stud 32 along the center axis direction of the stud 32. A through hole 321 is provided in an end surface of the tail end of the stud 32 to communicate with the sleeve hole 311, and the through hole 321 has a smaller diameter than the sleeve hole 311. The other end of the connecting shaft 5 is sleeved inside the sleeve hole 311 through the through hole 321. The limiting part 51 at the other end of the connecting shaft 5 cannot pass through the through hole 321, thereby restricting the stud 32 from coming out of the connecting shaft 5.

When the threaded sleeve 41 is rotated, the threaded sleeve 41 is limited in the axial direction where it is located. That is, the threaded sleeve can only rotate but cannot move up or down, so the stud 32 is forced to move up and down along the connecting shaft 5, and the stud 32 stops moving down after reaching a maximum stroke when it slides to the limiting part 51 of the connecting shaft 5. In this way, the connecting shaft 5 can not only prevent the push member 3 from coming out when the knob 4 is rotated, but also provide a guidance effect for the sliding of the stud 32, and a central position of the stud 32 can slide smoothly, so that smoother sliding adjustment is achieved, and the connection stability between the knob 4 and the push member 3 is also improved. In this embodiment, the limiting parts 51 at the two ends of the connecting shaft 5 are rivet structures, thereby achieving simple processing, simple assembly, and low cost. It can be understood that one end of the connecting shaft 5 connected inside the threaded sleeve 41 may alternatively not be fixedly connected to the threaded sleeve 41, as long as the stud 32 and the threaded sleeve 41 are slidably connected to the connecting shaft 5, so as to prevent the stud 32 from sliding off relative to the threaded sleeve 41.

The knob 4 further includes a screw cap 42 which fixedly covers one end of the threaded sleeve 41, and a gap is present between an inner circumferential surface of the screw cap 42 and a peripheral surface of the threaded sleeve 41.

A limiting ring 44 is arranged at one end of the threaded sleeve 41 and is mounted at one end of the end wall portion of the threaded sleeve 41. Preferably, the limiting ring 44 and the end wall portion may be integrated. The limiting ring 44 is rotatably mounted at an inner side of the foot side support member 1. The screw cap 42 is rotatably mounted at an outer side of the foot side support member 1. The screw cap 42 can be connected to the limiting ring 44 through a pin shaft which passes through the foot side support member 1.

Optimizing the structure of the knob 4 enhances the convenience of adjustment and improves the efficiency of mounting and dismounting, thereby not only improving the durability and stability of the corrector, but also bringing a more convenient and comfortable use experience for users. In order to improve the comfort in use, the hallux corrector provided by this disclosure further includes a joint pad 6 which is mounted on the push plate 31 and configured to abut against the metatarsophalangeal joint, where the joint pad 6 can be made of an elastic material, such as silicone.

In this embodiment, the foot side support member 1 is provided with a first connecting slot 100 for connecting a first bandage 7. The foot side support member 1 is worn on foot through the first bandage 7.

In this embodiment, the foot side support member 1 includes an inner side shell 11 and an outer side shell 12 which is detachably mounted on the inner side shell 11. The inner side shell 11 includes an inner arc-shaped plate 111 fitting with the metatarsal bone of a sole side, and a shaft sleeve seat 112 formed at one end of the inner arc-shaped plate 111. A periphery of the shaft sleeve seat 112 is provided with a step in an axial direction. The shaft sleeve seat 112 has a hollow axis hole 1121 running through the inner arc-shaped plate 111, and the axis hole 1121 is used for the threaded sleeve 41 to penetrate through. The hallux support member 2 is rotatably connected to a peripheral surface of the shaft sleeve seat 112, and is specifically rotatably clamped at a bottom side of the step of the shaft sleeve seat 112. The guide hole 10 is formed in the inner arc-shaped plate 111 and close to the shaft sleeve seat 112.

The outer side shell 12 includes an outer arc-shaped plate 121 connected to the inner arc-shaped plate 111, and a rotary drum 122 formed at one end of the outer arc-shaped plate 121. The outer arc-shaped plate 121 is fixedly mounted on the inner arc-shaped plate 111 to limit the hallux support member 2 in the axial direction. The rotary drum 122 includes a hollow drum portion. The rotary drum 122 sleeves the shaft sleeve seat 112. The threaded sleeve 41 extends into the axis hole 1121 of the shaft sleeve seat 112 and close to a top end of the shaft sleeve seat 112. The screw cap 42 rotatably sleeves the periphery of the rotary drum 122, and the screw cap 42 is fixedly connected to the limiting ring 44 through a pin shaft.

The threaded sleeve 41 and the stud 32 are screwed. The threaded sleeve 41 is driven to rotate by rotating the screw cap 42. Since the stud 32 and the limit guide member 33 are both fixedly arranged on the push plate 31, and the limit guide member 33 extends into the guide hole 10, so that when the threaded sleeve 41 rotates, the stud 32 does not rotate with the threaded sleeve 41, and the rotation of the threaded sleeve 41 forces the stud 32 to move in the axial direction.

It can be understood that the foot side support member 1 uses a structure in which the outer side shell 12 fits with the inner side shell 11, the inner side shell 11 is configured to be attached to a foot side, and the hallux support member 2 can be rotatably connected to the inner side shell 11. The outer side shell 12 is arranged to limit the axial position of the threaded sleeve 41 during rotation, that is, the threaded sleeve 41 does not move up or down relative to the foot side support member 1 during rotation. Specifically, a peripheral edge 1221 is formed at an inner top end of the rotary drum 122 on the outer side shell 12. A hole enclosed by the peripheral edge 1221 has a smaller diameter than the limiting ring 44, so that after the threaded sleeve 41 penetrates into the rotary drum 122, the screw cap 42 is connected to the threaded sleeve 41 through screws 43. Thus, the peripheral edge 1221 of the rotary drum 122 is clamped between a plate surface of the screw cap 42 and the limiting ring 44, and the screw cap 42 and the limiting ring 44 can rotate relative to the peripheral edge 1221. In this way, the threaded sleeve 41 does not move up or down relative to the foot side support member 1 during rotation. In order to improve the comfort in use, an outer surface of the inner side shell 11 is provided with an elastic gasket 13 made of a material such as silicone. In this embodiment, the screw cap 42 is connected to the limiting ring 44 of the threaded sleeve 41 through two screws 43 (equivalent to one pin shaft). It can be understood that the screws 43 run through a sleeve hole enclosed by the peripheral edge 1221, so that when the screw cap 42 rotates, the screws 43 are not hindered.

In this embodiment, the hallux support member 2 is provided with a second connecting slot 20 for connecting a second bandage 8. The hallux support member 2 is bundled on a hallux through the second bandage 8.

In this embodiment, the hallux support member 2 includes a curved plate 21 fitting with one side of the hallux, and a U-shaped groove 22 formed at one end of the curved plate 21. The U-shaped groove 22 clamps the peripheral surface of the shaft sleeve seat 112 to realize rotational connection. Thus, the hallux support member 2 is easily mounted on the shaft sleeve seat 112.

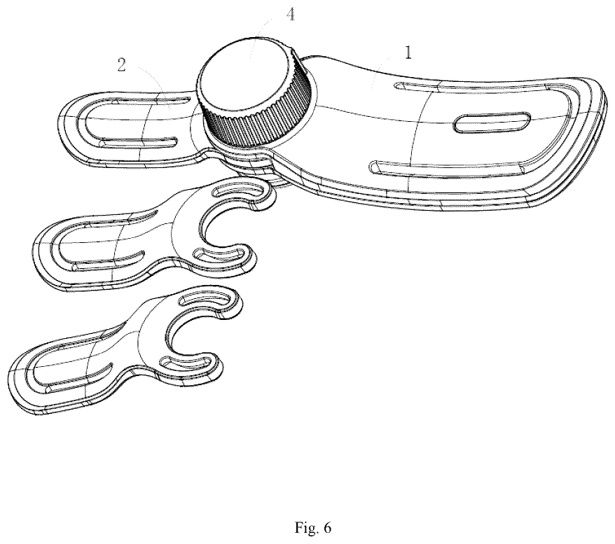

It can be understood that, referring to FIG. 6, various types of hallux support members 2 can be designed, with different fitting angles, a hallux support member 2 fitting with the shape of the hallux is selected according to a deviation degree of the hallux of the user, and the hallux support member 2 is inserted into the periphery of the shaft sleeve seat and can rotate around the periphery of the shaft sleeve seat.

When the hallux corrector is used, the rotation of the screw cap 42 forces the stud 32 to move along the axis direction, and the stud 32 moves together with the limit guide member 33 and the push plate 31. If the threaded sleeve 41 is rotated to push the stud 32 to move away from the threaded sleeve 41, since the push plate 31 abuts against the foot side, the foot side support member 1 and the hallux support member 2 are driven to move away from the foot side and to be tensioned. Since the foot side support member 1 is fixedly bundled on the sole and the foot side support member 1 has a large overall length, a degree of freedom of movement of the foot side support member 1 depends on the tightness of the bandage for bundling, or the foot side support member 1 has certain plastic deformation degree, so a degree of freedom of swing of the foot side support member 1 is small. However, the hallux has a certain degree of freedom of lateral swing, so the hallux can move laterally together with the hallux support member. Therefore, during adjustment, the foot side support member 1 and the hallux support member 2 can be integrally considered as a strip, which does a swing action by taking one end of the foot side support member as a fulcrum, that is, taking one end of the foot side support member abutting against a portion far away from the hallux as a support point. In this way, the hallux support plate is driven to move laterally with the hallux, with a maximum swing amplitude, thereby realizing distance adjustment of lateral movement of the hallux.

It is worth further explaining that this disclosure is not an easily conceivable replacement improvement of a deviation correction adjusting structure that uses a threaded knob structure appearing in the industry. It is required to overcome technical prejudice to achieve this disclosure. In the industry, the knob is designed as a stud, the push rod is provided with a central threaded hole, and the periphery of the push rod is provided with a limit guide structure. Such structure is easily conceivable by persons skilled in the art. If the knob is designed as a threaded hole structure, and the push rod is designed as a stud structure, the push rod is likely to fall off. In addition, since the periphery of the push rod is a thread, a limit guide structure cannot be arranged, which is not conducive to guidance. However, in this disclosure, the knob is designed as a threaded hole structure, and the push rod is designed as a stud structure, which is achieved by creative efforts. In this disclosure, in order to prevent the use of a structure in which a guide protrusion and a guide groove are snapped with each other so as to prevent occurrence of stuck during operation, the hallux corrector herein is designed to prevent the problem that in the industry, the push rod is provided with a threaded hole in the center, thus the peripheral wall of the push rod has a small thickness, and accordingly the push rod is not durable. In conclusion, the hallux corrector provided by this disclosure provides a high-efficiency and comfortable correction scheme for users through the beneficial effects of personalized adaptation, accurate and adjustable correction strength, high wearing comfort, simple operation and the like, and thus is expected to be widely applied and popularized in the field of medical auxiliary instruments.

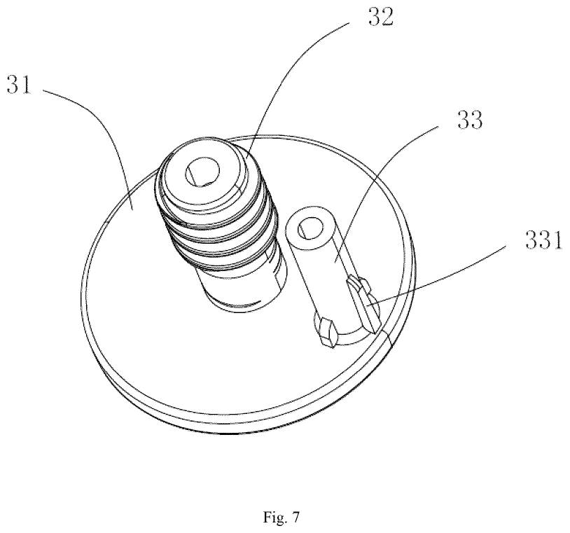

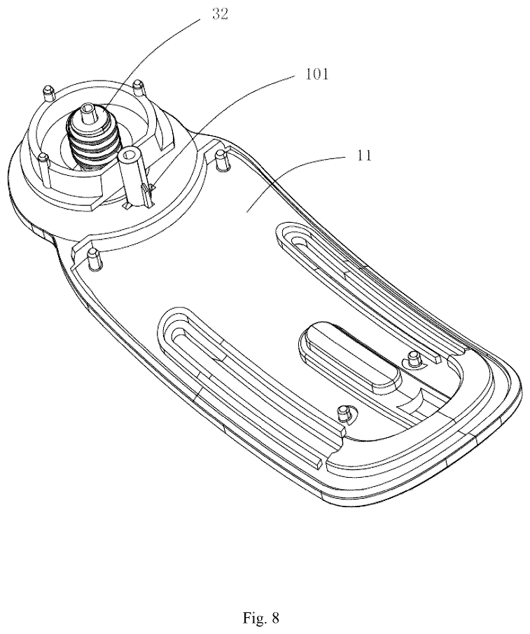

Referring to FIG. 7 and FIG. 8, further, in order to enhance the strength of the limit guide member 33, a plurality of reinforcing ribs 331 are formed on the periphery of the limit guide member 33, the reinforcing ribs 331 are integrally connected to the push plate 31, and correspondingly, corresponding cuts 101 are formed on a peripheral side of the guide hole 10 on the inner arc-shaped plate 111 for the matching reinforcing ribs 331 to pass through.

Any combination of various embodiments of this disclosure should be regarded as the disclosure of this disclosure as long as it does not depart from the idea of this disclosure. Within the scope of the technical concept of this disclosure, various simple modifications of the technical solution and any combination of different embodiments that do not depart from the idea created by this disclosure should all fall within the scope of protection of this disclosure.

What is claimed is:

1. A hallux corrector, comprising:
a foot side support member, capable of being worn on a sole and also capable of abutting against one side of the sole;
a hallux support member, capable of being worn on a hallux and also capable of abutting against an outer side surface of the hallux, wherein the hallux support member is rotatably connected to one end of the foot side support member;
a push member, wherein the push member comprises a push plate, a stud, a limit guide member, and a knob; the stud and the limit guide member are arranged on the push plate; and
the knob is rotatably mounted at a rotary joint between the foot side support member and the hallux support member; the knob comprises a threaded sleeve; the threaded sleeve is screwed to the stud; the limit guide member slidably penetrates into the foot side support member; the push plate and the threaded sleeve are respectively located at two sides of the foot side support member; the push plate abuts against a metatarsophalangeal joint of a foot side; a position of the threaded sleeve is defined at one side of the foot side support member through the rotary threaded sleeve; when the stud moves axially relative to the threaded sleeve, a distance from the threaded sleeve, the foot side support member and the hallux support member as a whole to the push plate changes; and the hallux support member, together with the hallux, changes in position relative to the push plate, so as to correct a position of the hallux.

2. The hallux corrector according to claim 1, wherein the foot side support member is provided with a guide hole, and the limit guide member slidably penetrates into the guide hole.

3. The hallux corrector according to claim 1, further comprising a connecting shaft, wherein the connecting shaft penetrates into the threaded sleeve and the stud; the stud is capable of sliding relative to the connecting shaft; two ends of the connecting shaft are each provided with a limiting part; and each of the limiting parts is configured to limit the threaded sleeve and the stud from leaving from the connecting shaft.

4. The hallux corrector according to claim 3, wherein one end of the connecting shaft extends into the threaded sleeve and is fixedly connected to one end of the threaded sleeve; the push plate is provided with a sleeve hole at a position corresponding to a center axis of the stud; the sleeve hole runs through the push plate; the sleeve hole extends along the center axis of the stud; an end surface of the stud is provided with a through hole; the through hole is in communication with the sleeve hole; the through hole has a smaller diameter than the sleeve hole; and the through hole is used for prevent pass of the limiting parts.

5. The hallux corrector according to claim 2, wherein the knob comprises a screw cap; one end of the threaded sleeve is provided with a limiting ring; the limiting ring is rotatably mounted at an inner side of the foot side support member; the screw cap is rotatably mounted at an outer side of the foot side support member; the screw cap is fixedly connected to the limiting ring through a screw; and the screw penetrates into the foot side support member.

6. The hallux corrector according to claim 5, wherein the foot side support member comprises an inner side shell and an outer side shell; the inner side shell comprises an inner arc-shaped plate and a shaft sleeve seat; the inner arc-shaped plate fits with metatarsal bone at a sole side; the shaft sleeve seat is formed at one end of the inner arc-shaped plate; the hallux support member is rotatably connected to a peripheral surface of the shaft sleeve seat; the outer side shell comprises an outer arc-shaped plate and a rotary drum; the rotary drum is formed at one end of the outer arc-shaped plate; the outer arc-shaped plate is fixedly mounted on the inner arc-shaped plate; and the rotary drum sleeves the shaft sleeve seat to axially limit the hallux support member.

7. The hallux corrector according to claim 6, wherein the shaft sleeve seat has an axis hole; the threaded sleeve extends into the axis hole; the screw cap rotatably sleeves the periphery of the rotary drum; an inner top end of the rotary drum is provided with a peripheral edge; a hole enclosed by the peripheral edge has a smaller diameter than the limiting ring; and the peripheral edge is clamped between the screw cap and the limiting ring.

8. The hallux corrector according to claim 6, wherein the hallux support member comprises a curved plate and a U-shaped groove; the curved plate fits with one side of the hallux; the U-shaped groove is formed at one end of the curved plate; and the U-shaped groove is snapped into the peripheral surface of the shaft sleeve seat to realize rotational connection.

9. The hallux corrector according to claim 6, further comprising a joint pad, wherein the pad is mounted on the push plate; the joint pad is configured to abut against the metatarsophalangeal joint; and an outer surface of the inner side shell is provided with an elastic gasket.

10. The hallux corrector according to claim 6, wherein the periphery of the limit guide member is provided with a plurality of reinforcing ribs; the reinforcing ribs are integrally connected to the push plate; a peripheral side of the guide hole is provided with a cut; and the reinforcing ribs pass through the cut.

11. The hallux corrector according to claim 1, wherein there are a plurality of types of foot side support members, with different fitting angles, and a corresponding foot side support member is selectively inserted into the periphery of the shaft sleeve seat according to a deviation degree of the hallux.
